(19) United States
(12) Patent Application Publication
(10) Pub. No.: US 2026/0057965 A1
(43) Pub. Date: Feb. 26, 2026

(54) TARGETED, AUTOMATED PRIMER AND PROBE RETRIEVAL: SYSTEMS AND METHODS FOR GENERATING QPCR ASSAYS

(71) Applicant: MRIGlobal, Kansas City, MO (US)

(72) Inventors: Phillip Evert Davis, Frederick, MD (US); Colin Webber Price, Falls Church, VA (US); Joseph Albert Russell, Frankford, DE (US)

(21) Appl. No.: 19/307,606

(22) Filed: Aug. 22, 2025

Related U.S. Application Data
(60) Provisional application No. 63/686,605, filed on Aug. 23, 2024.

(51) Int. Cl. G16B 25/20; G16B 15/10; G16B 30/00; G16B 40/20

(52) U.S. Cl. CPC G16B 25/20; G16B 15/10; G16B 30/00; G16B 40/20

(57) ABSTRACT

The present disclosure relates to an integrated system for generating optimized primer probe pair design for one or more quantitative polymerase chain reaction (qPCR) assays using a sequence alignment free design approach. The system includes a processor; and a computer-readable medium storing instructions which, when executed by the processor, cause the processor to: receive data including a genomic dataset; generate one or more k-mers from the genomic dataset; cluster the one or more k-mers to identify one or more targeted genomic regions; generate a plurality of primer-probe pair candidates corresponding to the one or more targeted genomic regions, wherein the one or more primer-probe pairs include degenerate primers; and perform one or more in silico operations using the one or more primers, wherein the one or more in silico operations comprise at least one of primer-probe pair optimization, specificity testing, secondary structure analysis, and in silico PCR simulation.

### CROSS-REFERENCE TO RELATED APPLICATION

This application claims the benefit of priority to U.S. Provisional Patent Application No. 63/686,605, filed on Aug. 23, 2024, entitled “TARGETED, AUTOMATED PRIMER AND PROBE RETRIEVAL: SYSTEMS AND METHODS FOR GENERATING QPCR ASSAYS,” the entire disclosure of which is hereby incorporated by reference.

### BACKGROUND

In molecular biology, the design of primers plays an important role in amplifying specific regions of DNA or RNA using techniques like quantitative polymerase chain reaction (qPCR). Primers are short, single-stranded DNA sequences are carefully crafted to bind to complementary target sequences, initiating the process of DNA synthesis. Effective primer design involves considerations such as sequence specificity of a primer, melting temperature (Tm) of a primer, and potential secondary structures formed by a primer, to ensure efficient and specific amplification. This process may be used for various analysis purposes, including, but not limited to, a detection of genetic variations, a study of gene expression patterns, and an identification of disease markers, potentially facilitating advancements in both research and diagnostic applications.

### BRIEF SUMMARY OF THE DISCLOSURE

The present disclosure generally relates to systems and methods for optimizing primer pairs for targeted sequences or genomic regions. Specifically, certain embodiments of the present disclosure may relate to systems and methods for designing primers for targeted sequences of genetic material pertaining to viruses, other pathogens (e.g., fungi or bacteria), and humans.

According to a first aspect, the present disclosure may include a system for generating one or more quantitative polymerase chain reaction (qPCR) assays comprising: a processor; and a computer-readable medium storing instructions which, when executed by the processor, cause the processor to: receive data including a genomic dataset; generate one or more k-mers from the genomic dataset; cluster the one or more k-mers to identify one or more targeted genomic regions; generate a plurality of primer-probe pair candidates corresponding to the one or more targeted genomic regions, wherein the one or more primer-probe pairs include degenerate primers; and perform one or more in silico operations using the one or more selected primer-probe pairs, wherein the one or more in silico operations comprise at least one of primer-probe pair optimization, specificity testing, secondary structure analysis, and in silico PCR simulation.

According to a second aspect of the first aspect or any other aspect, wherein the primer-probe optimization includes determining whether a melting temperature of the primer-probe pair is within an acceptable range.

According to a third aspect of the first aspect or any other aspect, wherein the primer-probe optimization includes determining whether a GC content of the primer-probe pair is within an acceptable range.

According to a fourth aspect of the first aspect or any other aspect, wherein the specificity testing includes determining an inclusivity value and an exclusivity value of the qPCR assay.

According to a fifth aspect of the first aspect or any other aspect, wherein the computer-readable medium stores instructions which, when executed by the processor, further cause the processor to: curate the data by removing one or more outlier sequences from the data.

According to a sixth aspect of the fifth aspect or any other aspect, wherein the data is clustered using one or more machine learning algorithms.

According to a seventh aspect of the first aspect or any other aspect, wherein the genomic dataset includes data on genetic material from one or more viruses.

According to an eighth aspect of the seventh aspect or any other aspect, wherein the one or more viruses include at least one of SARS-CoV-2 and Mpox.

According to a ninth aspect of the first aspect or any other aspect, wherein the data includes data stored in the NCBI database.

According to a tenth aspect of the present disclosure, a method for generating one or more primers corresponding to one or more targeted genomic regions comprising: receiving data including a genomic dataset; generating one or more k-mers from the genomic dataset; clustering the one or more k-mers to identify one or more targeted genomic regions; generating a plurality of primer-probe pair candidates corresponding to the one or more targeted genomic regions; selecting one or more primer-probe pairs from the plurality of primer-probe pair candidates, wherein the one or more primer-probe pairs include degenerate primers; and performing one or more in silico operations using the one or more selected primer-probe pairs, wherein the one or more in silico operations comprise at least one of primer-probe pair optimization, specificity testing, secondary structure analysis, and in silico PCR simulation.

According to an eleventh aspect of the tenth aspect or any other aspect, wherein the primer-probe optimization includes determining whether a melting temperature of the primer-probe pair is within an acceptable range.

According to a twelfth aspect of the tenth aspect or any other aspect, wherein the primer-probe optimization includes determining whether a GC content of the primer-probe pair is within an acceptable range.

According to a thirteenth aspect of the tenth aspect or any other aspect, wherein the specificity testing includes determining an inclusivity value and an exclusivity value of a quantitative polymerase chain reaction (qPCR) assay using the primer-probe pair.

According to a fourteenth aspect of the tenth aspect or any other aspect, further comprising curating the data by removing one or more outlier sequences from the data.

According to a fifteenth aspect of the present disclosure, a computer-implemented method for generating one or more primers corresponding to one or more targeted genomic regions comprising: displaying a first window containing data including at least one genomic dataset within a graphical user interface on a computer screen; displaying a second window comprising a plurality of icons within the graphical user interface, wherein the plurality of icons include at least one of a k-mer identification icon, a k-mer generation icon, a k-mer clustering icon, a primer-probe candidate generation icon, and an in silico operation icon; and generating one or more primers corresponding to one or more targeted genomic regions by: selecting the data from the first window; selecting the k-mer generation icon and utilizing a k-mer generation tool to generate one or more k-mers within the genomic dataset; selecting the k-mer clustering icon and utilizing a clustering tool to cluster the one or more generated k-mers to identify one or more targeted genomic regions; selecting the primer probe-candidate generation icon and utilizing a primer-probe candidate generation tool to generate a plurality of primer-probe pair candidates corresponding to the one or more targeted genomic regions; wherein the one or more primer-probe pairs include degenerate primers; and selecting the in silico operation icon and utilizing an in silico operation tool to perform one or more in silico operations using the one or more primers, wherein the one or more in silico operations comprise at least one of primer-probe pair optimization, specificity testing, secondary structure analysis, and in silico PCR simulation.

According to a sixteenth aspect of the fifteenth aspect or any other aspect, wherein the primer-probe optimization includes determining whether a melting temperature of the primer-probe pair is within an acceptable range.

According to a seventeenth aspect of the fifteenth aspect or any other aspect, wherein the primer-probe optimization includes determining whether a GC content of the primer-probe pair is within an acceptable range.

According to an eighteenth aspect of the fifteenth aspect or any other aspect, wherein the specificity testing includes determining an inclusivity value and an exclusivity value of a quantitative polymerase chain reaction (qPCR) assay using the primer-probe pair.

According to a nineteenth aspect of the fifteenth aspect or any other aspect, further comprising curating the data by removing one or more outlier sequences from the data.

According to a twentieth aspect of the fifteenth aspect or any other aspect, wherein: the second window further comprises a data curation icon; and generating one or more primers corresponding to one or more targeted genomic regions further comprises selecting the data curation icon and utilizing a data curation tool to curate the data by removing one or more outlier sequences from the data.

According to twenty-first aspect of the first aspect or any other aspect, wherein the genomic dataset includes data on genetic material from one or more pathogens but is agnostic of genetic material origin (e.g., human material).

According to a twenty-second aspect of the first aspect or any other aspect, wherein the computer-readable medium stores instructions which, when executed by the processor, further cause the processor to: output one or more generated primer-probe pairs predicted to maximize amplification efficiency and specificity while minimizing secondary structure formation wherein the system is configured to optimize primer-probe selection based on predicted thermodynamic properties and reaction kinetics.

According to a twenty-third aspect of the tenth aspect or any other aspect, further comprising outputting one or more generated primer-probe pairs predicted to maximize amplification efficiency and specificity while minimizing secondary structure formation.

### BRIEF DESCRIPTION OF THE DRAWINGS

The foregoing Summary and the following Detailed Description will be better understood when read in conjunction with the appended drawings. In the drawings:

FIG. 1 is a schematic diagram of a process for generating one or more qPCR assays, performing in silico PCR testing, and evaluating the qPCR assays.

FIG. 2 is a schematic diagram illustrating an integrated system for generating one or more qPCR assays, according to one embodiment of the present disclosure.

FIG. 3 is a method flow diagram of a method for generating one or more qPCR assays and evaluating the one or more qPCR assays using one or more in silico operations, according to one embodiment of the present disclosure.

FIG. 4 is a schematic diagram illustrating examples of in silico operations that can be performed using the system of FIG. 2 or the method of FIG. 3, according to one embodiment of the present disclosure.

FIG. 5 is a schematic of a graphical user interface for a computing device that enables a user to perform the operations of the system of FIG. 2, which may include the use of an integrated genomic analysis tool having a cohesive workflow that supports the design of both general primers and degenerate primers, according to one embodiment of the present disclosure.

FIG. 6 is a graph illustrating a detection sensitivity for Mpox virus comparing qPCR assays generated by the system of FIG. 2 and qPCR assays that are publicly available, according to one embodiment of the present disclosure.

FIG. 7 is a graph illustrating a detection sensitivity for SARS-CoV 2 virus comparing qPCR assays generated by the system of FIG. 2 and qPCR assays that are publicly available, according to one embodiment of the present disclosure.

FIG. 8 is a graph illustrating a limit of detection for SARS-CoV 2 virus comparing qPCR assays generated by the system of FIG. 2 and qPCR assays that are publicly available, according to one embodiment of the present disclosure.

### DETAILED DESCRIPTION

The current public health enterprise may respond to emerging infectious disease outbreaks with outdated and ineffective diagnostic assays, thereby failing to provide an effective diagnostic test in a time of a crisis. The speed of generating diagnostic assays in response to emerging infections disease outbreaks is naturally prioritized, and thus, the most recently published assays (i.e., from scientific literature) are often chosen for diagnostic surveillance in the public health response. However, this commonly results in ineffective, non-optimized, and outdated assays that do not encompass a pathogen's rapidly evolving taxonomic clade. Further, outdated assays do not provide a reliable diagnostic test to the public. Thus, it is desirable to provide a rapid de novo assay design process that can conform to a rapid public health response timeline.

Furthermore, it is desirable to integrate various genomic analysis tools into a cohesive workflow that supports the design of both general primers (i.e., a short, specific DNA sequence used in PCR to initiate the amplification of a known target region by binding to its complementary sequence on the template nucleic acid) and degenerate primers (i.e., a primer containing multiple possible sequences at certain positions to accommodate variations in the target sequence, allowing amplification of related but variable sequences across different samples or species).

As discussed herein and by non-limiting example, qPCR assays may be generated to target current infectious disease outbreaks such as SARS-CoV 2 and Mpox. However, the systems and methods described herein are not limited to these examples and may be used for primer design agnostic of generic material origin (e.g., markers for healthy human tissue as well as non-viral pathogens).

As discussed herein, a “k-mer” is substring of a biological sequence, such as a nucleic acid sequence or a protein sequence, of length k that is extracted from a longer nucleic acid sequence (e.g., DNA, RNA) or protein sequence. In certain embodiments, a k-mer is used in bioinformatics for computational genomics and sequence analysis by breaking down larger sequences into more manageable segments.

Systems and methods are disclosed herein to develop quantitative PCR assays. As used herein, a qPCR assay refers to a technique used to detect and measure the quantity of a specific nucleic acid target in a sample. This may include traditional PCR methods, PCR methods that incorporate a quantitative element that allows for real-time monitoring of the DNA amplification process, or other amplification techniques.

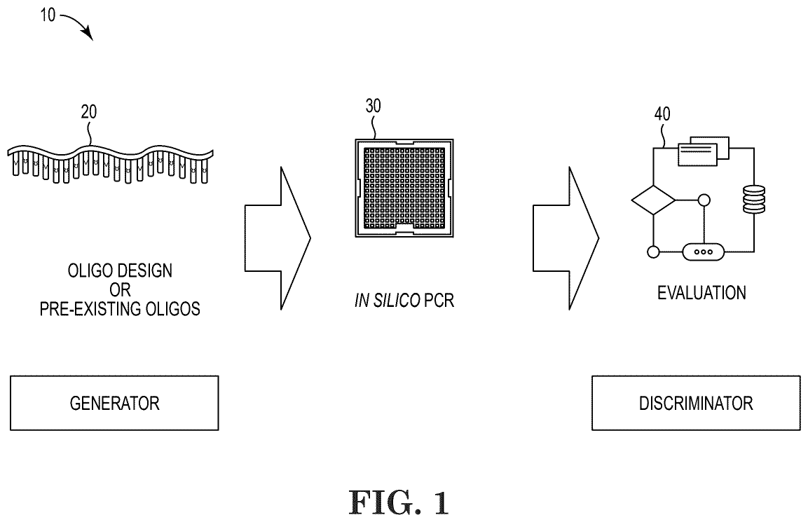

According to one embodiment, one or more systems and methods described herein utilize a software tool named Targeted Automated Primer and Probe Retriever (TAPPR), which is designed for bioinformatics applications. The primary functionality of TAPPR is to facilitate the design of specific primers for targeted genomic regions, particularly for use in molecular biology research and diagnostics. The process involves searching for conserved sequences (i.e., sequences that remain largely unchanged across different species or strains and correspond to functionally important regions of a genome) within a genomic dataset, clustering these sequences to identify target groups, and designing primers that can be used to detect and amplify these target regions in PCR experiments. TAPPR's utility spans various genomic scales, from large genomes to smaller, single-gene organisms. This functionality of TAPPR is illustrated and described in further detail with respect to FIGS. 1-5.

As illustrated and described below, the systems described herein (including TAPPR) can be structured modularly to allow one or more users to manage various components of the primer design process separately or in combination depending on the specific needs of the experiment.

The modular components include scripts for counting kmers, clustering sequences, generating primer candidates, and performing in silico PCR simulations. A modular design enables flexibility, as users can modify or extend each part of the workflow independently. For instance, the clustering and counting processes for k-mers can be adjusted for different types of genomic data, enhancing the tool's adaptability to diverse research requirements. The modular structure of one or more embodiments of the system described herein are illustrated and described in further detail with respect to FIGS. 1-5.

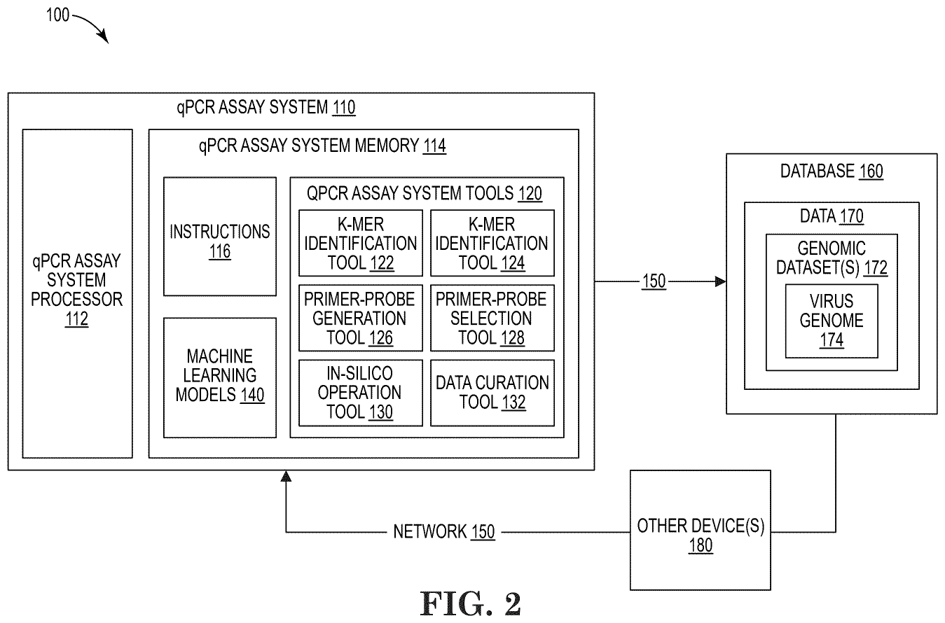

FIG. 1 is a schematic diagram of a process 10 including a series of steps 20, 30, 40 for generating one or more qPCR assays 20, performing in silico qPCR assays 30, and evaluating the qPCR assays 40. The example process 10 is described and illustrated in further detail below with reference to this figure and FIGS. 2-4.

The example process 10 includes a step 20 of designing one or more oligos or oligonucleotides, such as primers or probes, or using one or more pre-existing oligos. An oligo is a short, single-stranded nucleic acid sequence configured to bind to a complementary sequence. Oligos may be used in target identification processes like PCR, e.g., as primers, or for DNA or RNA sequencing. In certain embodiments, the oligo consists of 15-30 nucleotides and serves as a primer or probe to initiate or detect specific genetic sequences.

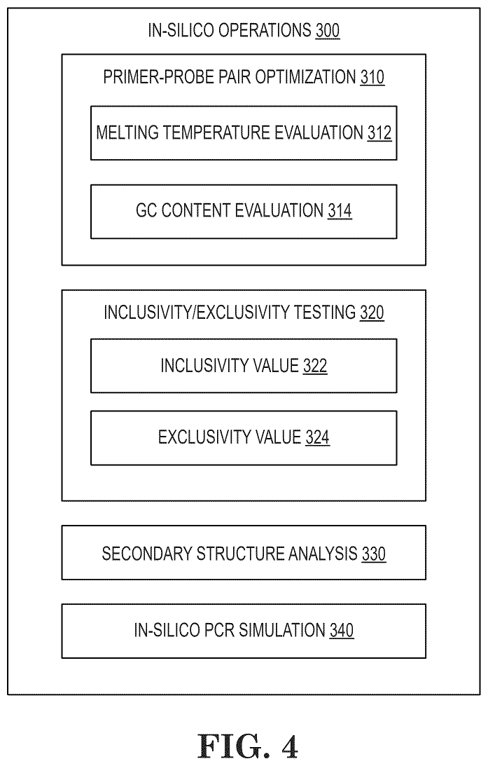

Oligos can be designed, or a pre-existing oligo can be selected, based on a plurality of criteria. The proper design or selection of oligos in qPCR analysis is desirable to ensure the accuracy and efficiency of qPCR techniques. The plurality of criteria can include, for example, the length of the oligo (e.g., 15-30 nucleotides in range), a sequence that is complementary to a target DNA/RNA sequence, the GC content (i.e., the percentage of guanine (G) and cytosine (C) bases in the oligo), the melting temperature of the oligo (e.g., primer), and minimizing the formation of oligo secondary structures (e.g., hairpins and dimers), and oligo specificity to a target sequence without cross-reactivity to non-target sequences. In silico operations for evaluating these design characteristics are illustrated and described in further detail with respect to FIG. 4.

The example process 10 includes a step 30 of performing one or more in silico operations (i.e., experiments or simulations conducted using computer models or computer data analysis rather than performing biological studies in a traditional laboratory setting). In silico operations are desirable because they are less expensive than traditional laboratory experiments or clinical trials, large datasets can be processed quickly and efficiently, in silico operations provide the ability to predict outcomes based on models, and in silico operations minimize ethical issues of utilizing animal or human subjects in clinical trials. Examples of in silico operations for evaluating these design characteristics are illustrated and described in further detail with respect to FIG. 4.

The example process includes a step 40 of evaluating the efficacy of qPCR assays to ensure the qPCR assays will perform as desired in physical experimentation. In certain embodiments, examples of evaluations can include, by non-limiting example, blast analysis and secondary structure prediction, melting point calculation, an efficiency assessment to predict how well the primers will amplify the target DNA, and cross-reactivity checks that evaluate a potential for non-specific amplification.

FIG. 2 is a schematic diagram illustrating a system for generating one or more qPCR assays, according to one embodiment of the present disclosure.

Systems and software, e.g., implemented on a non-transitory computer-readable medium, for performing the methods discussed herein are within the scope of embodiments of the present disclosure.

Embodiments of the present disclosure may thus utilize a special purpose or general-purpose computing system including computer hardware, such as, for example, one or more processors and system memory. As discussed herein, the system 100 includes a qPCR assay system 110. The qPCR assay system 110 includes a qPCR assay system processor 112 and a qPCR assay system memory 114.

Embodiments within the scope of the present disclosure also include physical and other computer-readable media for carrying or storing computer-executable instructions 116 and/or data structures, including applications, tables, data, libraries, or other modules used to execute functions or direct selection or execution of other modules. Such computer-readable media can be any available media that can be accessed by a general purpose or special purpose computer system. Computer-readable media that store computer-executable instructions (or software instructions) are physical storage media. Computer-readable media that carry computer-executable instructions are transmission media. Thus, by way of example, and not limitation, embodiments of the present disclosure can include at least two distinctly different kinds of computer-readable media, namely physical storage media or transmission media. Combinations of physical storage media and transmission media should also be included within the scope of computer-readable media.

Both physical storage media and transmission media may be used temporarily to store or carry software instructions in the form of computer readable program code that allows performance of embodiments of the present disclosure. Physical storage media may further be used to persistently or permanently store such software instructions. Examples of physical storage media include physical memory (e.g., RAM, ROM, EPROM, EEPROM, etc.), optical disk storage (e.g., CD, DVD, HDDVD, Blu-ray, etc.), storage devices (e.g., magnetic disk storage, tape storage, diskette, etc.), flash or other solid-state storage or memory, or any other non-transmission medium which can be used to store program code in the form of computer-executable instructions or data structures and which can be accessed by a general purpose or special purpose computer, whether such program code is stored as or in software, hardware, firmware, or combinations thereof.

A network 150 or “communications network” may generally be defined as one or more data links that enable the transport of electronic data between computer systems and/or modules, engines, and/or other electronic devices. When information is transferred or provided over a communication network or another communications connection (either hardwired, wireless, or a combination of hardwired or wireless) to a computing device, the computing device properly views the connection as a transmission medium. Transmission media can include a communication network and/or data links, carrier waves, wireless signals, and the like, which can be used to carry desired program or template code means or instructions in the form of computer-executable instructions or data structures and which can be accessed by a general purpose or special purpose computer. As illustrated and described in FIG. 1, the network 150 enables transport of electronic data between the qPCR assay system 110, one or more databases 160, and other device(s) 180, which are illustrated and described in further detail below.

Further, upon reaching various computer system components, program code in the form of computer-executable instructions or data structures can be transferred automatically or manually from transmission media to physical storage media (or vice versa). For example, computer-executable instructions or data structures received over a network or data link can be buffered in memory (e.g., RAM) within a network interface module (NIC), and then eventually transferred to computer system RAM and/or to less volatile physical storage media at a computer system. Thus, it should be understood that physical storage media can be included in computer system components that also (or even primarily) utilize transmission media.

In certain embodiments, the qPCR Assay system memory 114 stores one or more qPCR assay system tools 120. As shown in FIG. 2, the qPCR system tools 120 can include, by non-limiting example, a k-mer generation tool 122, a k-mer clustering tool 124, a primer generation tool 126, a primer-probe selection tool 128, an in silico operation tool 130, and a data curation tool 132. In certain embodiments, the qPCR assay system tools 120 utilize one or more machine learning models 140 to complete one or more functions as illustrated and described below.

Together, the k-mer generation tool 122 and the k-mer clustering tool 124 may be referred to as the “k-mer identification tool.” The k-mer identification tool is used to identify specific regions within a target sequence to design primers. In certain embodiments, the specific regions are short sequences (e.g., 18-25 nucleotides long) that bind to target DNA with a desired level of specificity. In certain embodiments, the k-mer identification tool analyzes one or more k-mers to ensure the primers will specifically bind to target sequences while avoiding any non-target sequences to minimize off-target DNA amplification.

The k-mer generation tool 122 divides the input sequence(s) into a string(s) containing k bases, where k is set as the primer length or k is set as a range of primer lengths. The primer length (k) may be selected based on the desired specificity and the nature of the sequences. An input sequence (e.g., from a genomic dataset) having a length (l) can be divided into l−k+1 k-mers. For example, for the input sequence CTGACTGAG, with k set to 4, the input sequence can be divided into 6 k-mers: CTGA, TGAC, GACT, ACTG, CTGA, and TGAG.

The k-mers generated from the input sequence (in the example above, the 9 k-mers generated from the input sequence) may be counted and the frequency of each k-mer may be determined. An analysis of k-mer frequency and k-mer set operations may be performed. The k-mer frequency analysis may be based on the principle that conserved regions of a gene(s) are the same or very similar across multiple genomes of the same clade. As such, it is inferred that a k-mer set that results from the intersection of k-mers from all input sequences should be derived from such conserved regions (when the k-value is greater than a certain length). Thus, the frequency of a k-mer or a set of k-mers generated from the input sequence, e.g., a genome(s), a genomic region(s), or a sequence(s), can be used as an identifier or signature of the input sequence. Comparing k-mer sets generally requires less computation time than using sequence alignment analysis. Generally, the subset of intersecting k-mer(s) are selected for further analysis or processing or used directly to design primer(s). Additionally or alternatively, the k-mers generated from the input sequence may be counted and processed using a k-mer clustering tool 124.

A k-mer clustering tool 124 may organize the generated k-mers) into meaningful groups to identify patterns or regions of interest within the sequences. Clustering k-mers allows for one to focus on clusters that are specific to a disease being studied while also reducing the number of k-mers to consider, which simplifies the process of identifying unique or highly specific k-mers. In certain embodiments, the k-mer clustering tool 124 clusters one or more k-mers by calculating a similarity metric to measure the similarity between the k-mers. In certain embodiments, the similarity metric may include one or more of a Hamming Distance, a Jaccard Index, an edit distance, or other form of distance matrix. In certain embodiments, the k-mer identification tool is used to select one or more primer target sequences in the biological sequence(s) (e.g., DNA sequence) of one or more pathogens of interest (e.g., the SARS CoV-2 and Mpox viruses). The k-mer identification tool may process and analyze data 170 that includes one or more genomic datasets 172 storing one or more virus genomes, genomic regions, or sequences 174 (e.g., the SARS CoV-2 and Mpox virus genomes). This data 170 can be received by the qPCR assay system 110 from one or more databases 160 via the network 150. In certain embodiments, the database 160 includes data 170 stored in the National Center for Biotechnology Information's (NCBI's) public database that stores consortiums of DNA and transcript (RNA).

In certain embodiments, the k-mer identification tool can analyze data 170 collected from one or more databases 160 and perform a quality check (e.g., ensuring the collected sequences are free of contaminants or errors, removing redundant sequences, filtering sequences based on length), before generating the one or more k-mers. In some embodiments, the k-mers identified by the k-mer identification tool may be mapped to a reference sequence to validate that the identified k-mers are present in the sequence being studied (e.g., from Sars-CoV-2 or from Mpox).

Furthermore, in certain embodiments, the k-mer clustering tool 124 clusters one or more k-mers by utilizing a machine learning model 140. In certain embodiments, the machine learning algorithm is a supervised machine learning algorithm or an unsupervised machine learning algorithm.

Supervised learning is a type of machine learning where the machine learning model 140 is trained on a labeled dataset, meaning that each training example is paired with an output label. The goal is to learn a mapping from inputs to outputs to make predictions on new, unseen data. For example, the machine learning models 140 may be trained to label a dataset where k-mers are associated with known characteristics, such as whether they are within a target pathogen sequence (e.g., Sars-CoV-2 or Mpox). The machine learning model 140 can then be trained using one or more features to distinguish between k-mers that are relevant to produce a desirable qPCR assay and those that are not. A trained machine learning model 140 can then be used to predict a likelihood that a new k-mer belongs to a target class based on criteria provided for the k-mer clusters and cluster the k-mers based on the predicted labels.

An unsupervised model is trained on data without explicit labels or output values. The goal is to identify patterns, groupings, or structures within the data, such as clustering similar items or reducing data dimensions. In certain embodiments, the machine learning models 140 may be trained using unsupervised machine learning, which includes a training phase and a clustering phase. In the training phase, a machine learning model 140 is trained to generate k-mers from genomic datasets 172 without using predefined labels. The machine learning model 140 can then compute a similarity between the generated k-mers to assess how similar they are to one another and evaluate the effectiveness of the algorithm (e.g., a clustering algorithm) to group k-mers. This process can be repeated until the machine learning model 140 is sufficiently trained to cluster the k-mers effectively. Once the machine learning model 140 is effectively trained, it can be applied to cluster one or more k-mers and analyze the clusters to identify k-mers that are within a target pathogen sequence. In certain embodiments, the machine learning model 140 may perform these steps automatically or assist a user in performing these steps.

The primer generation tool 126 generates primer-probe pairs that are specific to target sequences in PCR assays. In certain embodiments, the primer generation tool 126 is configured to use clustering results from the k-mer clustering tool 124 to identify k-mers that are within a target pathogen sequence (e.g., Sars-CoV-2 or Mpox). Relevant clusters can be selected that include k-mers that are within a target pathogen sequence and are not within non-target sequences to generate primers that have high inclusivity and high exclusivity metrics). The primer generation tool 126 can generate one or more primers (e.g., a forward and reverse primer) that flank a specific region of the target sequence. The primer generation tool 126 may generate primers that are specific to the target sequence, have an optimal length, and exhibit minimal self-dimerization or cross-dimerization with other primers. The primer generation tool 126 can also generate one or more probes that bind in close proximity to the forward or reverse primer without overlapping with the primer-binding site(s).

Once a primer-probe pair is generated, the primer-probe pair can be validated using one or more in silico operations using the in silico operation tool 130. The in silico operation tool 130 performs one or more in silico operations (300) that are illustrated and described in further detail with respect to FIG. 4.

In certain embodiments, the qPCR assay system tools 120 include a data curation tool 132. The data curation tool 132 is configured to receive data 170 from a database 160 and curate the data 170 to ensure analysis performed using the data is accurate, relevant, and in a suitable format, e.g., FASTA. In certain embodiments, this can include the use one or more machine learning models 140 that utilize supervised or unsupervised learning to retrieve data 170, assess the quality of the data 170, clean the data, filter and normalize the data, perform any other pre-processing steps and validate the accuracy of the data 170.

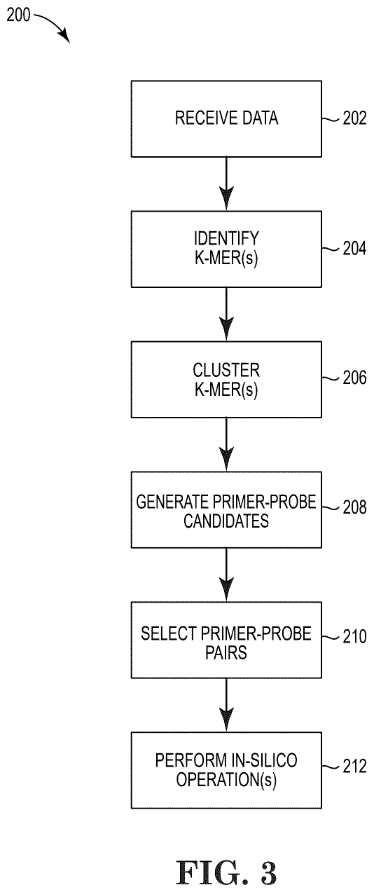

FIG. 3 is a method flow diagram of a method 200 for generating one or more primers for qPCR assays and evaluating the one or more primers using one or more in silico operations 300, in accordance with the present disclosure.

The method 200 includes a step of receiving data 170 including a genomic dataset 172. In certain embodiments, and as illustrated and described in further detail above with respect to FIG. 2, data 170 can be received from one or more databases 160. In certain embodiments, and as illustrated and described in further detail above with respect to FIG. 2, the data curation tool 132 may be used to curate the data 170 by removing one or more outlier sequences from the data 170. The method 200 includes a step 204 of identifying one or more k-mers. In certain embodiments, one or more k-mers are identified using the k-mer identification tool, which may include the k-mer generation tool 122 and the k-mer clustering tool 124, as illustrated and described above with reference to FIG. 1. In certain embodiments, one or more conserved regions of sequence(s) are used to identify k-mer(s). A process by which one or more k-mers are identified can include, by non-limiting example, dividing an input sequence(s) (from the data 170) into k-mers, counting and/or clustering the k-mers, for example, measuring the similarity between the k-mers, performing a frequency analysis (i.e., calculating how frequently each k-mer appears in the input sequence), performing intersection or difference operations on k-mer sets, and selecting one or more k-mers having frequencies that are greater than a predetermined threshold).

The method 200 includes a step 208 of generating one or more primer-probe pair candidates corresponding to the one or more targeted genomic regions. In certain embodiments, the primer-probe candidates are generated using the primer generation tool 126 as illustrated and described above with reference to FIG. 1. Furthermore, in certain embodiments, the one or more primer-probe pairs include degenerate primers. A degenerate primer is a mixture of similar primer sequences that incorporate variations at specific positions to account for the degeneracy of the genetic code.

The method 200 includes a step 212 of performing one or more in silico operations using the one or more primers generated in step 208. In certain embodiments, the one or more in silico operations are performed using the in silico operation tool 130 as illustrated and described above with reference to FIG. 1. Furthermore, various non-limiting examples of in silico operations that can be performed using the in silico operations tool 130 in step 212 are illustrated and described in further detail with respect to FIG. 4.

FIG. 4 is a schematic diagram illustrating example in silico operations 300 that can be performed using the system 100 of FIG. 2 or the method 200 of FIG. 3, in accordance with the present disclosure.

The in silico operations 300 include primer-probe pair optimization 310. Primer-probe optimization is desirable to ensure a generated PCR assay is specific, efficient, and includes desirable properties. In certain embodiments, the primer probe optimization 310 includes a melting temperature evaluation 312 and a GC content evaluation 314.

The melting temperature is the temperature at which half of the DNA strands are in a double-helix state and half are in a single-strand state. The melting temperature can be used to evaluate specificity, as primers with a melting temperature that is too low may bond nonspecifically and primers with a melting point that is too high may not bind efficiently to target DNA. In certain embodiments, the melting temperature evaluation 312 includes determining whether a melting temperature of the primer-probe pair is within an acceptable range.

The GC content evaluation 314 refers to a determination of the percentage of guanine (G) and cytosine (C) nucleotides in a primer or probe. It is desirable to measure the GC content of a primer because GC pairs contribute to the specificity of the primer-probe pairs by ensuring they bind strongly and specifically to a target region without forming secondary structures or dimers. GC pairs may increase the binding strength and stability of a primer because they form three hydrogen bonds compared to two bonds formed in adenine (A) and thymine (T) pairs. Ideally, the GC content is within a range of about 40% to about 60%). In certain embodiments, the GC content evaluation 314 includes determining whether a GC content of the primer-probe pair is within a selected range.

In certain embodiments, the in silico operations 300 include an assessment of the inclusivity/exclusivity 320 of the qPCR assay. Inclusivity and exclusivity testing 320 is used to evaluate the performance and specificity of the qPCR assays to ensure they reliably detector include all of the target pathogen strains of interest (inclusivity), while also avoiding cross-reactivity with non-target organisms (exclusivity). Specifically, the inclusivity/exclusivity testing 320 includes determining an inclusivity value 322 and an exclusivity value 324 of the qPCR assay. An inclusivity value 322 determines a degree of which the assay can detect all relevant variants or strains of a target pathogen to ensure the assay is effective across not only a desired variation, but also other genetic variations of the pathogen. The exclusivity value 324 determines a degree of which the assay can detect a target pathogen without cross-reacting with non-target organisms or pathogens.

In certain embodiments, the inclusivity value 322 is expressed as a percentage indicating the number of target strains that are successfully detected by the qPCR assay out of a total number of target strains tested in the assay. In certain embodiments, the exclusivity value 324 is expressed as a percentage indicating the number of non-target organisms or strains not detected by the assay out of a total number of non-target organisms tested.

In certain embodiments, the inclusivity value 322 and/or exclusivity value is obtained in silico by selecting a representative collection (including various strains or genetic variants of a target pathogen) of sequences from a genetic database, e.g., NCBI, and evaluating the primer, primer-probe pair, or amplicon sequence for similarities/differences between targets/non-targets.

In certain embodiments, the in silico operations 300 include secondary structure analysis 330. Secondary structure analysis 330 relates to the study of a spatial arrangement of nucleotides in DNA or RNA sequences. Various secondary structures for nucleic acids can include, by non-limiting example, hairpins, loops, and other structures. Secondary structure analysis 330 can include the use of one or more computational prediction tools for predicting the formation of secondary structures of a primer or probe.

In certain embodiments, the in silico operations 300 include one or more in silico PCR simulations 340. The in silico PCR simulation 340 is a computation method used to predict the performance of a qPCR assay based on data rather than physical lab experiments. In certain embodiments, the in silico PCR simulation 340 may utilize other in silico operations 300, for example, in silico primer design. In silico PCR simulation may include in silico PCR validation, which involves using the designed primers/primer-probe pairs in the PCR simulation and evaluating the performance of the assay, for example, the specificity, and optionally providing one or more optimization recommendations for improving PCR performance.

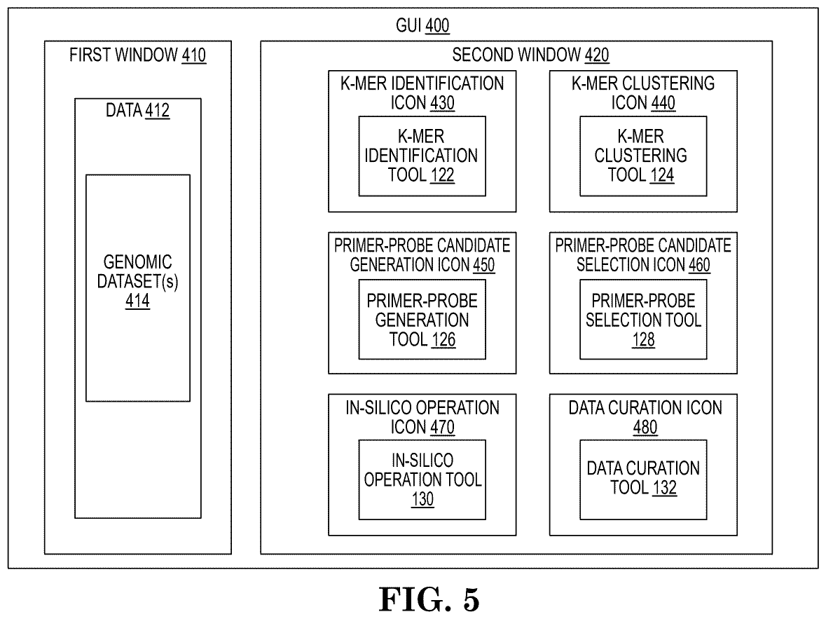

FIG. 5 is a schematic of a graphical user interface 400 on a computing device that enables a user to perform the operations of the system 100 of FIG. 2, in accordance with the present disclosure.

In certain embodiments, the graphical user interface 400 can include a first window 410 and a second window 420 on a computer screen. The first window 410 can include data 412 including at least one genomic dataset 414. The second window 420 can include a plurality of icons 430, 440, 450, 460, 470, 480 including at least one of a k-mer identification icon 430, a k-mer generation icon, a k-mer clustering icon 440, a primer-probe candidate generation icon 450, and an in silico operation icon 470. Furthermore, in certain embodiments, the second window 420 includes a data curation icon 480.

The k-mer identification icon 430, k-mer generation icon, k-mer clustering icon 440, primer-probe candidate generation icon 450, in silico operation icon 470, and the data curation icon 480 provide access to the k-mer identification tool, k-mer generation tool, k-mer clustering tool 124, primer generation tool 126, in silico operation tool 130 and the data curation tool 132, respectively. The k-mer identification tool, k-mer generation tool, k-mer clustering tool 124, primer generation tool 126, in silico operation tool 130 and the data curation tool 132 are illustrated and described in further detail with respect to FIG. 1.

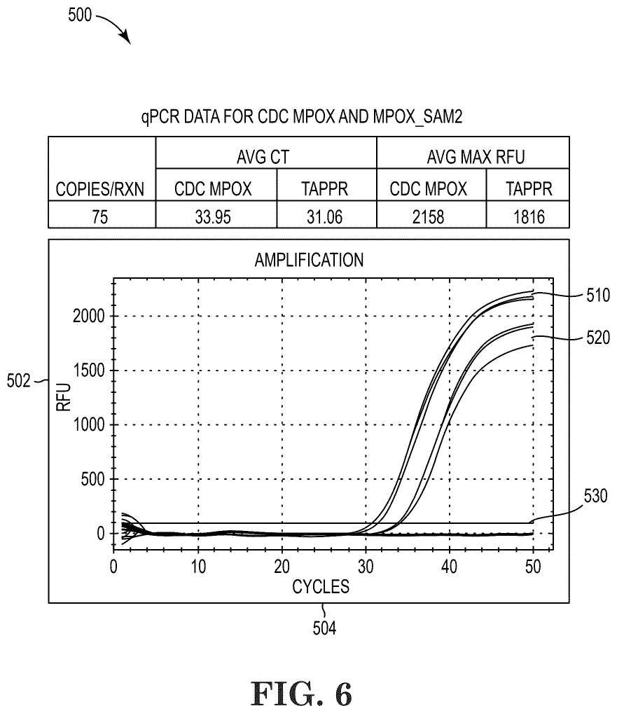

FIG. 6 is a graph 500 illustrating a detection sensitivity of qPCR assays generated by the system 100 of FIG. 2 and qPCR assays that are publicly available, both assays for detecting Mpox virus.

The graph 500 includes a measurement of the number of qPCR cycles performed (cycles) 504 versus the relative fluorescence units (RFU) 502 measured during qPCR. In qPCR, the measured fluorescence is proportional to the amount of amplicon/PCR product and the change in fluorescence over time may be used to calculate the amount of amplicon/PCR product produced in each cycle.

As shown in the graph 500, the measured fluorescence (RFU) of a qPCR 510 assay for detecting Mpox developed in accordance with the present disclosure is greater than that of a publicly available Mpox qPCR assay 520 from the Center for Disease Control (CDC) at fewer cycles (i.e., the measured fluorescence (RFU 502) of the qPCR assay 510 developed in accordance with the present disclosure increases exponentially at an earlier cycle count as compared to the publicly available qPCR assay 520), which indicates that the assay 510 developed in accordance with the present disclosure is effective at detecting a target sequence at lower concentrations of the target sequence. Thus, the assay 510 developed in accordance with the present disclosure enables increased expression of the amplicon/PCR product (as indicated by the greater peak intensity of fluorescence) and earlier detection of the Mpox virus as compared to the publicly available qPCR assay 520. The graph 500 further includes a negative control 530, which lacks the target sequence and does not show an increase in fluorescence with as the cycle count increases.

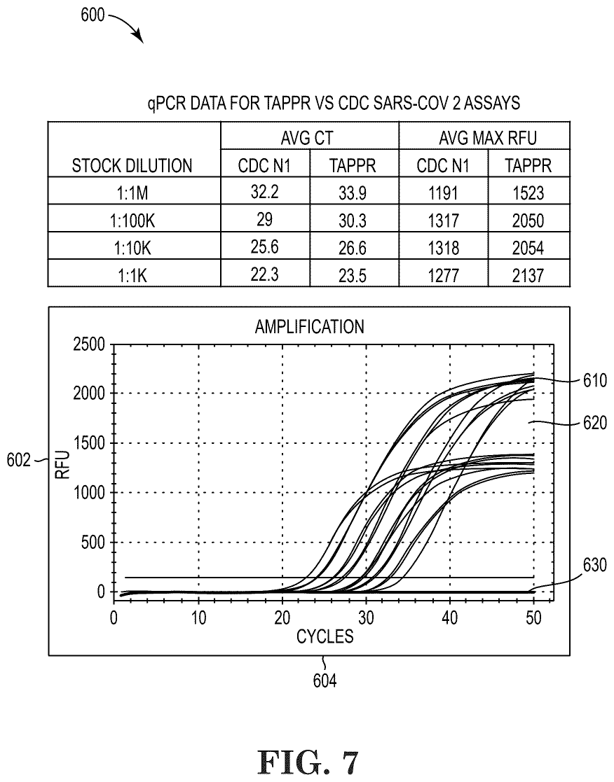

FIG. 7 is a graph 600 illustrating a detection sensitivity of qPCR assays generated by the system 100 of FIG. 2 and qPCR assays that are publicly available, both assays for detecting SARS-CoV 2 virus.

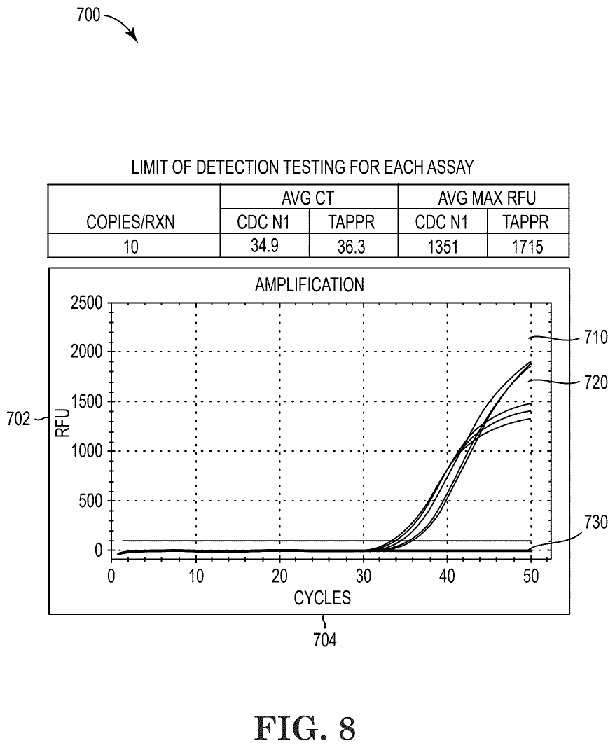

The graph 600 includes a measurement of the number of qPCR cycles performed (cycles) 604 versus the relative fluorescence units (RFU) 602 measured during qPCR. Although the graph 600 indicates that a publicly available SARS-CoV 2 virus qPCR assay 520 from the Center for Disease Control (CDC) provides slightly earlier detection of Sars-Cov-2 virus than a qPCR 610 assay for detecting SARS-CoV 2 virus developed in accordance with the present disclosure, the peak intensity of fluorescence for the qPCR 610 assay developed in accordance with the present disclosure is greater than that of the publicly available (from the CDC) qPCR assay 620. FIG. 8 is a graph illustrating a limit of detection of the qPCR assays generated by the system 100 of FIG. 2 and qPCR assays that are publicly available, both assays for detecting SARS-CoV 2 virus.

The graph 700 includes a measurement of the number of qPCR cycles performed (cycles) 704 versus the relative fluorescence units (RFU) 702 measured during qPCR. As shown in the graph, at 10 genomic copies per reaction, both the qPCR assay 710 developed in accordance with the present disclosure and the publicly available qPCR assay 720 were able to detect the target sequence, which indicates that the sensitivity and inclusivity of the qPCR assay 710 developed in accordance with the present disclosure is similar to the sensitivity and inclusivity of an assay that has been stringently optimized by researchers globally (the publicly available qPCR from the CDC).

One or more specific embodiments of the present disclosure are described herein. These described embodiments are examples of the presently disclosed techniques. Additionally, to provide a concise description of these embodiments, not all features of an actual embodiment may be described in the specification. It should be appreciated that in the development of any such actual implementation, as in any engineering or design initiative, numerous embodiment-specific decisions will be made to achieve the developers' specific goals, such as compliance with system-related constraints, which may vary from one embodiment to another. Moreover, it should be appreciated that such a development effort might be complex and time consuming, but would nevertheless be a routine undertaking of design, fabrication, and manufacture for those of ordinary skill having the benefit of this disclosure.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, references to “one embodiment” or “an embodiment” of the present disclosure are not intended to be interpreted as excluding the existence of additional embodiments that also incorporate the recited features. For example, any element described in relation to an embodiment herein may be combinable with any element of any other embodiment described herein. Numbers, percentages, ratios, or other values stated herein are intended to include that value, and other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

The present disclosure may be embodied in other specific forms without departing from its spirit or characteristics. The described embodiments are to be considered as illustrative and not restrictive. The scope of the disclosure is, therefore, indicated by the appended claims rather than by the foregoing description. Changes that come within the meaning and range of equivalency of the claims are to be embraced within their scope.

### CONCLUSION

The foregoing description of the exemplary embodiments has been presented only for the purposes of illustration and description is not intended to be exhaustive or to limit the compositions, systems, and methods herein to the precise forms disclosed. Many modifications and variations are possible considering the above teachings.

The embodiments were chosen and described in order to explain the principles of the technology discussed herein and their practical application to enable others skilled in the art to utilize the various embodiments and with various modifications as are suited to the particular use contemplated. Alternative embodiments will become apparent to those skilled in the art to which the present technologies pertain without departing from their spirit and scope.

What is claimed is:

1. A system for generating one or more quantitative polymerase chain reaction (qPCR) assays comprising:
a processor; and
a computer-readable medium storing instructions which, when executed by the processor, cause the processor to:
receive data including a genomic dataset;
generate one or more k-mers from the genomic dataset;
cluster the one or more k-mers to identify one or more targeted genomic regions;
generate a plurality of primer-probe pair candidates corresponding to the one or more targeted genomic regions, wherein the one or more primer-probe pairs include degenerate primers; and
perform one or more in silico operations using the one or more selected primer-probe pairs, wherein the one or more in silico operations comprise at least one of primer-probe pair optimization, specificity testing, secondary structure analysis, and in silico PCR simulation.

2. The system of claim 1, wherein the primer-probe optimization includes determining whether a melting temperature of the primer-probe pair is within an acceptable range.

3. The system of claim 1, wherein the primer-probe optimization includes determining whether a GC content of the primer-probe pair is within an acceptable range.

4. The system of claim 1, wherein the specificity testing includes determining an inclusivity value and an exclusivity value of the qPCR assay.

5. The system of claim 1, wherein the computer-readable medium stores instructions which, when executed by the processor, further cause the processor to:
curate the data by removing one or more outlier sequences from the data.

6. The system of claim 5, wherein the data is clustered using one or more machine learning algorithms.

7. The system of claim 1, wherein the genomic dataset includes genetic material from one or more pathogens.

8. The system of claim 7, wherein the one or more pathogens include at least one of SARS-CoV-2 and Mpox.

9. The system of claim 1, wherein the computer-readable medium stores instructions which, when executed by the processor, further cause the processor to:
output one or more generated primer-probe pairs predicted to maximize amplification efficiency and specificity while minimizing secondary structure formation wherein the system is configured to optimize primer-probe selection based on predicted thermodynamic properties and reaction kinetics.

10. A method for generating one or more primers corresponding to one or more targeted genomic regions comprising:
receiving data including a genomic dataset;
generating one or more k-mers from the genomic dataset;
clustering the one or more k-mers to identify one or more targeted genomic regions;
generating a plurality of primer-probe pair candidates corresponding to the one or more targeted genomic regions;
selecting one or more primer-probe pairs from the plurality of primer-probe pair candidates, wherein the one or more primer-probe pairs include degenerate primers; and
performing one or more in silico operations using the one or more selected primer-probe pairs, wherein the one or more in silico operations comprise at least one of primer-probe pair optimization, specificity testing, secondary structure analysis, and in silico PCR simulation.

11. The method of claim 10, wherein the primer-probe optimization includes determining whether a melting temperature of the primer-probe pair is within an acceptable range.

12. The method of claim 10, wherein the primer-probe optimization includes determining whether a GC content of the primer-probe pair is within an acceptable range.

13. The method of claim 10, wherein the specificity testing includes determining an inclusivity value and an exclusivity value of a quantitative polymerase chain reaction (qPCR) assay using the primer-probe pair.

14. The method of claim 10, further comprising curating the data by removing one or more outlier sequences from the data.

15. The method of claim 10, further comprising outputting one or more generated primer-probe pairs predicted to maximize amplification efficiency and specificity while minimizing secondary structure formation.

16. A computer-implemented method for generating one or more primers corresponding to one or more targeted genomic regions comprising:
displaying a first window containing data including at least one genomic dataset within a graphical user interface on a computer screen;
displaying a second window comprising a plurality of icons within the graphical user interface, wherein the plurality of icons include at least one of a k-mer identification icon, a k-mer generation icon, a k-mer clustering icon, a primer-probe candidate generation icon, and an in silico operation icon; and
generating one or more primers corresponding to one or more targeted genomic regions by:
selecting the data from the first window;
selecting the k-mer generation icon and utilizing a k-mer generation tool to generate one or more k-mers within the genomic dataset;
selecting the k-mer clustering icon and utilizing a clustering tool to cluster the one or more generated k-mers to identify one or more targeted genomic regions;
selecting the primer probe-candidate generation icon and utilizing a primer-probe candidate generation tool to generate a plurality of primer-probe pair candidates corresponding to the one or more targeted genomic regions;
wherein the one or more primer-probe pairs include degenerate primers; and
selecting the in silico operation icon and utilizing an in silico operation tool to perform one or more in silico operations using the one or more primers, wherein the one or more in silico operations comprise at least one of primer-probe pair optimization, specificity testing, secondary structure analysis, and in silico PCR simulation.

17. The method of claim 16, wherein the primer-probe optimization includes determining whether a melting temperature of the primer-probe pair is within an acceptable range.

18. The method of claim 16, wherein the primer-probe optimization includes determining whether a GC content of the primer-probe pair is within an acceptable range.

19. The method of claim 16, wherein the specificity testing includes determining an inclusivity value and an exclusivity value of a quantitative polymerase chain reaction (qPCR) assay using the primer-probe pair.

20. The method of claim 16, wherein:
the second window further comprises a data curation icon; and
generating one or more primers corresponding to one or more targeted genomic regions further comprises selecting the data curation icon and utilizing a data curation tool to curate the data by removing one or more outlier sequences from the data.
